(19) United States
(12) Patent Application Publication
(10) Pub. No.: US 2020/0294644 A1
(43) Pub. Date: Sep. 17, 2020

(54) SOLID PHARMACEUTICAL AGENT DOSAGE FORM DISPENSING AND BIOMETRIC DATA ACQUISITION DEVICE

(71) Applicant: Sumner Bluffs, LLC, Tea, SD (US)

(72) Inventors: Tony A. Hagen, Sioux Falls, SD (US); Tim Hagen, Elk Grove, CA (US); Jake Zastrow, Sioux Falls, SD (US)

(21) Appl. No.: 16/685,587

(22) Filed: Nov. 15, 2019

Related U.S. Application Data
(63) Continuation of application No. 15/521,849, filed on Apr. 25, 2017, now abandoned.
(60) Provisional application No. 62/068,648, filed on Oct. 25, 2014.
(60) Provisional application No. 62/191,972, filed on Jul. 13, 2015.

(51) Int. Cl. G16H 20/13; G16H 10/60; G16H 40/63

(52) U.S. Cl. CPC G16H 20/13; Y02A 90/26; G16H 40/63; G16H 10/60

(57) ABSTRACT

Embodiments of the present disclosure generally relate to devices, methods and systems for dispensing solid forms of an agent to a subject. In some embodiments an agent disclosed herein can include a pharmaceutical, biological or other solid agent for dispensing to a subject in need thereof. In certain embodiments, the present disclosure describes devices, methods and systems for biometric data acquisition and monitoring before, during, and/or after dispensing solid dosage forms of an agent to a subject. In some embodiments, these devices can be portable and/or handheld devices.

### RELATED APPLICATIONS

The instant application claims the benefit of U.S. Provisional Patent Application Ser. No. 62/068,648, filed Oct. 25, 2014, and U.S. Provisional Patent Application Ser. No. 62/191,972, filed Jul. 13, 2015. These applications are incorporated herein by reference in their entirety for all purposes.

### FIELD

Embodiments of the present disclosure generally relate to devices, methods and systems for dispensing solid forms of an agent to a subject. In some embodiments an agent disclosed herein can include a pharmaceutical, biological or other solid agent for dispensing to a subject in need thereof. In certain embodiments, the present disclosure describes devices, methods and systems for biometric data acquisition and monitoring before, during, and/or after dispensing solid dosage forms of an agent to a subject. In some embodiments, these devices can be portable and/or handheld devices.

### BACKGROUND

The concept of personalized medicine is changing the healthcare landscape throughout the world. Personalized medicine is an emerging field that uses various diagnostic tools (e.g., genetic markers, biometric data) to help determine which medical treatments and procedures will be best for a given patient. By combining this personalized diagnostic information with a patient's medical records and individual needs, personalized medicine allows physicians and patients to develop targeted prevention and treatment plans. The goal of personalized medicine is to provide the right treatment in the right dose to the right patient at the right time.

Although great progress has been made, the goals of personalized medicine have not yet been fully realized. For example, there is a paucity of currently available drug delivery devices with the capability to administer safely and effectively one or more pharmaceutical agents to a patient. Prescription medications and over-the-counter drugs are administered to patients in various forms, and this typically requires a different device for each mode of administration. Additionally, physicians typically must not only rely on their patients to adhere to their medical instructions after leaving the clinical setting, they must also trust that their patients are accurately reporting information regarding their treatment. The ability for patients to have more control over the delivery of their medication, while at the same time providing physicians with meaningful and accurate biometric and diagnostic data during treatment would greatly augment the overall goals of personalized medicine and lead to better patient outcomes.

### SUMMARY

Embodiments of the present disclosure include a solid pharmaceutical and/or other agent dosage form dispensing and biometric data acquisition device including a housing unit, a power source, a processor and optionally, memory. Other embodiments herein can include a loading cartridge having a plurality of rotatable storage chambers for storing and dispensing one or more solid pharmaceutical dosage forms, and a prime mover operably coupled to the loading cartridge and a dispensing element associated or found within the dispensing and biometric data acquisition device. In some aspects, activation of the dispensing element actuates a prime mover to eject or dispense one or more solid pharmaceutical and/or other agent dosage forms from the loading cartridge.

Some embodiments can also include at least one scanner operatively coupled to the processor. The processor can, for example, verify the identity of a subject based upon information obtained by the at least one scanner. Other embodiments can include at least one biometric sensor for sensing biometric data of the subject. The biometric data can be sensed and acquired during at least one of prior to, during, and/or after dispensing the one or more solid pharmaceutical dosage forms to the subject, and the biometric data can be stored in memory. Other embodiments can also include at least one data transfer port.

In some aspects of the present invention, the device can further include an airflow port and an image acquisition device centrally aligned with the airflow port. In accordance with these embodiments, the image acquisition device comprises a lens, an image sensor and signal wires which operatively connect the image acquisition device to the processor. Embodiments disclosed herein can also include at least one visual indicator or audio indicator that emits at least one light signal or sound operatively coupled to the image acquisition device and facing the same direction as the lens. In some aspects, the device can include an airflow port accessory module coupled to the airflow port to facilitate the inhalation and exhalation of the user's breath into the airflow port.

In other embodiments, a device can further include at least one biometric sensor such as one or more of a temperature sensor, a galvanic skin response sensor, a pulse oximeter, a carbon dioxide sensor, an oxygen sensor, an optical sensor, an air flow velocity sensor, an air pressure sensor, a chemical sensor, and a global positioning system (GPS) sensor. In other aspects, the device further includes one or more peripheral module interfaces for coupling one or more peripheral modules to the device. The peripheral modules can include one or more of a blood pressure monitor, a blood glucose monitor, a CPAP machine, an electrocardiogram device, a battery, and a battery charger.

In some aspects, a scanner included in a device can be a fingerprint reader, a pressure sensor, and/or a radio-frequency identification (RFID) reader. In other aspects, the device can include Bluetooth™ hardware and software components. In yet other aspects of the invention, the processor included in the device can further include one or more software programs for operating a biometric sensor and for facilitating the acquisition of biometric data using a biometric sensor.

In other embodiments, the one or more solid pharmaceutical or other agent dosage form can be one or more of a pill, capsule, gel capsule, gummy, timed-release capsule, slow-dissolving capsule, tablet, caplet, patch, strip, and thin-film strip.

Embodiments of the present disclosure also include a system for dispensing a solid pharmaceutical or other dosage form to and acquiring biometric data from, a subject. The system can include: a housing unit, a power source, a processor and memory. Other embodiments can include a loading cartridge including a plurality of rotatable storage chambers for storing and dispensing one or more solid pharmaceutical agent dosage forms, and a prime mover operably coupled to the loading cartridge and a dispensing element. In some aspects, activation of the dispensing element actuates the prime mover to eject one or more solid pharmaceutical agent dosage forms from the loading cartridge. Embodiments can also include at least one scanner operatively coupled to the processor. The processor can be capable of verifying the identity of a subject based in part on information obtained by the scanner. Certain embodiments can also include at least one biometric sensor for sensing biometric data of the subject. The biometric data can be sensed and acquired during at least one of prior to, during, and/or after dispensing the one or more solid pharmaceutical dosage forms to the subject, and the biometric data can be stored in memory. Other embodiments can also include at least one data transfer port.

Some embodiments of the present disclosure can also include a method for dispensing a solid pharmaceutical agent dosage form to an authorized user. In accordance with these embodiments, the method can include scanning a biometric identifier of a user using a biometric data acquisition and solid pharmaceutical dosage form dispensing device; determining, using the biometric identifier, whether the user is approved to take a pre-determined (e.g. prescribed) dose of a solid pharmaceutical agent dosage form; and dispensing the dosage of the solid pharmaceutical agent dosage form from the solid pharmaceutical dosage form dispensing and biometric data acquisition device, if the user is approved to take the dosage of the solid pharmaceutical dosage form.

In some aspects, methods can include scanning a biometric identifier, including, but not limited to the following: a fingerprint pattern, an iris pattern, a retina pattern, a vocal pattern, a facial-feature pattern, a pore pattern, a thermal image pattern, or a blood vessel pattern.

In certain aspects of methods disclosed herein, determining whether a user is approved to take a dosage of a solid pharmaceutical dosage form can include matching the scanned biometric identifier to a stored biometric identifier, wherein the stored biometric identifier can be an approved user's biometric identifier; identifying if the solid pharmaceutical agent dosage form is included on a list of solid pharmaceutical agent dosage forms that are eligible to be taken by the user; identifying a recent time that the biometric identifier was scanned and the solid pharmaceutical dosage form was dispensed; and approving the dispensing of the solid pharmaceutical dosage form if a present time during which the biometric identifier is scanned is within an approved time period for dispensing the solid pharmaceutical dosage form, based on a recent time that the biometric identifier was scanned.

In some aspects, the method can include recording, on the solid pharmaceutical agent dosage form dispensing and biometric data acquisition device's memory, times that the solid pharmaceutical dosage form is dispensed. In some aspects, methods include transmitting a time that the dosage of the pharmaceutical dosage form was dispensed, the dosage amount that was dispensed, and identity of the solid pharmaceutical agent dosage form that was dispensed to an auxiliary electronic device. In other aspects, the method includes taking an image of the user, using an imaging device, when dispensing a dosage of the solid pharmaceutical agent dosage form, wherein the imaging device can be coupled to the biometric data acquisition and solid pharmaceutical agent dosage form dispensing device.

In some aspects, methods disclosed herein can include providing sensory feedback to the user. Sensory feedback can include, but is not limited to, visual cues, haptic feedback, or auditory feedback. In other aspects, methods can include measuring a biometric response to the dosage of the solid pharmaceutical dosage form. The biometric response can include, but are not limited to, a galvanic skin response, a blood oxygen level response, a body temperature response, a heartrate response, a perfusion index response, a blood pressure response, a retina response, an eye movement response, an inhalation velocity response, an inhalation pressure response, an inhalation volume response, an expiratory velocity response, an expiratory pressure response, an expiratory volume response, or an exhale chemical composition response.

In other aspects, methods can include transmitting the dosage of the solid pharmaceutical dosage form and the measured biometric response to an auxiliary electronic device. In other aspects, methods include dispensing a revised dosage of the solid pharmaceutical dosage form based on the measured biometric response.

### Definitions and Terms

As used herein, the terms “subject,” “user,” and/or “patient” can include humans and other animals or mammals that are in need of treatment and capable of using or have assisted use of devices and systems as described herein. Additionally, the terms “subject,” “user,” and/or “patient” can include humans and other mammals treated in any type of environment such as a clinical setting, non-clinical setting, experimental setting, etc.

The term “solid pharmaceutical dosage form,” “solid pharmaceutical agent dosage form” and derivatives thereof, as used herein, include but is not limited to, prescribed or otherwise regulated compounds in the form of pill, capsule, gel capsule, gummy, timed-release capsule, slow-dissolving capsule, tablet, caplet, patch, strip, and thin-film strip , mini-tablets, granules, beads, pellets, multiparticulates, spheroids, or combinations thereof. If the solid dosage form is a tablet, the tablet can be of any suitable shape such as round, spherical, or oval. A solid pharmaceutical dosage form may have a monolithic or a multilayered structure, and may contain active pharmaceutical ingredients, including prescription or non-prescription pharmaceutical agents, in both solid and liquid forms.

As used herein the terms “pharmaceutical,” “pharmaceutical agent,” “biological agent,” “biologic,” “monitored agent,” “agent” and “drug” can mean a pharmaceutically effective compound, and/or effective compound and/or the pharmaceutically acceptable salt of a pharmaceutically effective compound, used in the treatment of a disease or condition. For example, a pharmaceutical drug or agent contemplated herein can be used in the treatment of diseases such as asthma, bronchitis, emphysema, lung infection, cystic fibrosis, alpha-1 anti-trypsin (AAT) deficiency, chronic obstructive pulmonary disease (COPD), acute respiratory distress syndrome (ARDS), infant respiratory distress syndrome (IRDS), borderline personality disorder (BPD), and macrophage activation syndrome (MAS), among many other conditions. Useful pharmaceutical agents can be delivered via inhalation, injection, ingestion, by feeding tube, and/or sublingually, according to the present disclosure, but are not limited to only those listed in the present disclosure. Generally, the agents that can be delivered using the devices and systems of the present disclosure have been approved by the U.S. Food and Drug Administration. Other agents or drugs may be used in accordance with the devices and systems of the present disclosure; the agents listed in the present disclosure are not intended to be exhaustive.

The terms “determine,” “calculate,” and “compute,” and variations thereof, as used herein, are used interchangeably and include any type of methodology, process, mathematical operation or technique.

It is to be noted that the term “a” or “an” entity refers to one or more of that entity. As such, the terms “a” (or “an”), “one or more” and “at least one” can be used interchangeably herein. It is also to be noted that the terms “comprising,” “including,” and “having” can be used interchangeably.

As used herein, “at least one,” “one or more,” and “and/or” are open-ended expressions that are both conjunctive and disjunctive in operation. For example, each of the expressions “at least one of A, B and C,” “at least one of A, B, or C,” “one or more of A, B, and C,” “one or more of A, B, or C,” and “A, B, and/or C” means A alone, B alone, C alone, A and B together, A and C together, B and C together, or A, B and C together. When each one of A, B, and C in the above expressions refers to an element, such as X, Y, and Z, or class of elements, such as X₁-Xₙ, Y₁-Yₘ, and Z₁-Zₒ, the phrase can be intended to refer to a single element selected from X, Y, and Z, a combination of elements selected from the same class (e.g., X₁ and X₂) as well as a combination of elements selected from two or more classes (e.g., Y₁ and Zₒ).

The term “means” as used herein shall be given its broadest possible interpretation in accordance with 35 U.S.C. § 112(f). Accordingly, a claim incorporating the term “means” shall cover all structures, materials, or acts set forth herein, and all of the equivalents thereof. Further, the structures, materials or acts and the equivalents thereof shall include all those described in the summary, brief description of the drawings, detailed description, abstract, and claims themselves.

The term “computer-readable medium” as used herein refers to any storage and/or transmission medium that participate in providing instructions to a processor for execution. Such a medium can be commonly tangible and non-transient and can take many forms, including but not limited to, non-volatile media, volatile media, and transmission media and includes without limitation random access memory (“RAM”), read only memory (“ROM”), and the like. Non-volatile media includes, for example, NVRAM, or magnetic or optical disks. Volatile media includes dynamic memory, such as main memory. Common forms of computer-readable media include, for example, a floppy disk (including without limitation a Bernoulli cartridge, ZIP drive, and JAZ drive), a flexible disk, hard disk, magnetic tape or cassettes, or any other magnetic medium, magneto-optical medium, a digital video disk (such as CD-ROM), any other optical medium, punch cards, paper tape, any other physical medium with patterns of holes, a RAM, a PROM, and EPROM, a FLASH-EPROM, a solid state medium like a memory card, any other memory chip or cartridge, a carrier wave as described hereinafter, or any other medium from which a computer can read. A digital file attachment to e-mail or other self-contained information archive or set of archives is considered a distribution medium equivalent to a tangible storage medium. When the computer-readable media is configured as a database, it is to be understood that the database may be any type of database, such as relational, hierarchical, object-oriented, and/or the like. Accordingly, the disclosure can be considered to include a tangible storage medium or distribution medium and prior art-recognized equivalents and successor media, in which the software implementations of the present disclosure are stored. Computer-readable storage medium commonly excludes transient storage media, particularly electrical, magnetic, electromagnetic, optical, magneto-optical signals.

The term “module” as used herein refers to any known or later developed hardware, software, firmware, artificial intelligence, fuzzy logic, or combination of hardware and software that can be capable of performing the functionality associated with that element. Also, while the disclosure can be presented in terms of exemplary embodiments, it should be appreciated that individual aspects of the disclosure can be separately claimed.

“Radio-Frequency IDentification” (RFID) refers to the use of a wireless non-contact system that uses radio-frequency electromagnetic fields to transfer data from a tag attached to an object, for the purposes of automatic identification and/or tracking. Some tags require no battery and are powered and read at short ranges via magnetic fields (electromagnetic induction) (known as passive RFID tags). Others use a local power source and emit radio waves (electromagnetic radiation at radio frequencies) (known as active RFID tags). The tag contains electronically stored information which may be read from up to several meters away. Unlike a bar code, the tag does not need to be within line of sight of the reader and may be embedded in the tracked object.

It should be understood that every maximum numerical limitation given throughout this disclosure can be deemed to include each and every lower numerical limitation as an alternative, as if such lower numerical limitations were expressly written herein. Every minimum numerical limitation given throughout this disclosure can be deemed to include each and every higher numerical limitation as an alternative, as if such higher numerical limitations were expressly written herein. Every numerical range given throughout this disclosure can be deemed to include each and every narrower numerical range that falls within such broader numerical range, as if such narrower numerical ranges were all expressly written herein.

The preceding is a simplified summary of the disclosure to provide an understanding of some aspects of the disclosure. This summary is neither an extensive nor exhaustive overview of the disclosure and its various aspects, embodiments, and configurations. It is intended neither to identify key or critical elements of the disclosure nor to delineate the scope of the disclosure but to present selected concepts of the disclosure in a simplified form as an introduction to the more detailed description presented below. As will be appreciated, other aspects, embodiments, and configurations of the disclosure are possible utilizing, alone or in combination, one or more of the features set forth above or described in detail below.

### BRIEF DESCRIPTION OF THE DRAWINGS

The accompanying drawings are incorporated into and form a part of the specification to illustrate several examples of the present disclosure. These drawings, together with the description, explain the principles of the disclosure. The drawings simply illustrate preferred and alternative examples of how the disclosure can be made and used and are not to be construed as limiting the disclosure to only the illustrated and described examples. Further features and advantages will become apparent from the following, more detailed, description of the various aspects, embodiments, and configurations of the disclosure, as illustrated by the drawings

FIG. 1 is a representative block diagram of a system incorporating a pharmaceutical delivery and biometric monitoring device, according to one embodiment of the present disclosure.

FIG. 2 is a representative diagram of the top view of a pharmaceutical or other solid agent delivery and biometric monitoring device, according to one embodiment of the present disclosure.

FIG. 3 is a representative diagram of a side view of a pharmaceutical or other solid agent delivery and biometric monitoring device, according to one embodiment of the present disclosure.

FIG. 4 is a representative diagram of the bottom view of a pharmaceutical or other solid agent delivery and biometric monitoring device, according to one embodiment of the present disclosure.

FIG. 5 is a representative flow diagram of a method for authenticating a user using the pharmaceutical deliver and biometric monitoring device, according to one embodiment of the present disclosure.

FIG. 6 is a representative flow diagram illustrating a specific example of the method for authenticating a user using the pharmaceutical and biometric monitoring device described in FIG. 5.

### DETAILED DESCRIPTION

Embodiments of the present disclosure generally relate to devices and systems for dispensing solid pharmaceutical or other agent dosage forms to a subject. In certain embodiments, the present disclosure discloses devices, methods and systems for biometric data acquisition and monitoring before, during, and after dispensing solid pharmaceutical or other agent dosage forms to a subject.

Embodiments of the devices of the present disclosure can include three principal components: a scanner to verify and/or authenticate a user (e.g., a fingerprint scanner), a biometric sensor to acquire user biometric data (e.g., a pulse oximeter), and a pharmaceutical delivery component (e.g., an inhalation canister) to deliver a pharmaceutical, biological or other monitored agent to the user. In accordance with these embodiments, the devices of the present disclosure can be handheld, allowing an authenticated user or caregiver to deliver a pharmaceutical or biological agent or other monitored agent while acquiring user biometric data before, during and/or after administration of the pharmaceutical or biological agent or other solid agent. In some embodiments, the devices of the present disclosure can also facilitate transfer of user biometric data to an authorized caregiver, healthcare professional or physician, which can be used by the caregiver, healthcare provider or physician to evaluate accurately the user's condition and provide more effective treatment options.

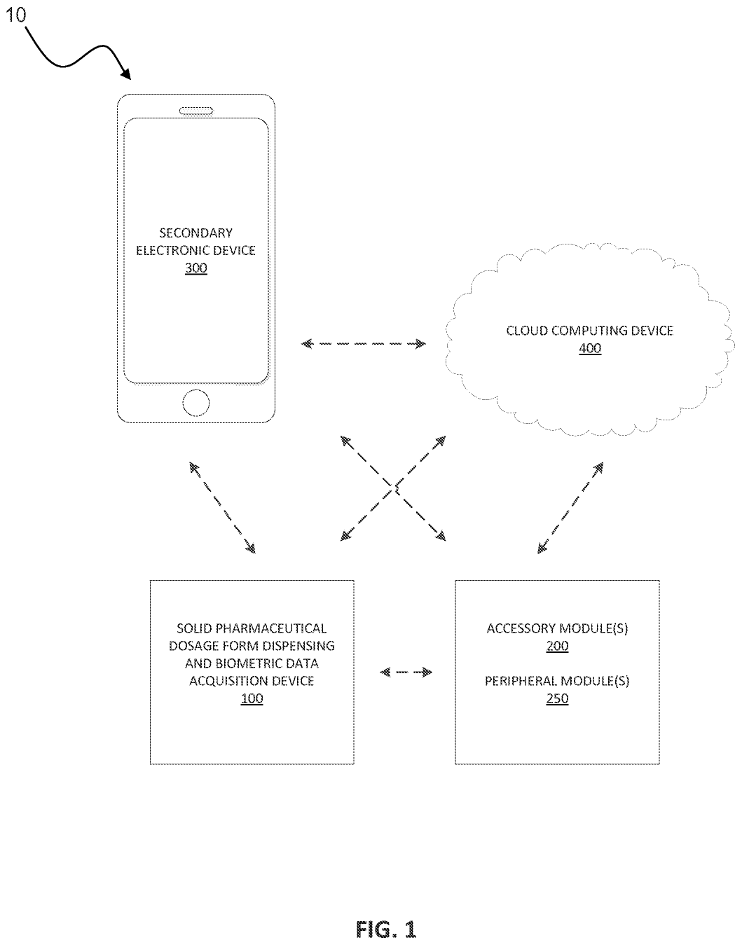

FIG. 1 is a representative block diagram of a system 10 incorporating the solid pharmaceutical or other agent dosage form dispensing and biometric data acquisition device 100, according to one embodiment of the present disclosure. The system 10 includes a solid pharmaceutical dosage form dispensing and biometric data acquisition device 100, one or more accessory modules 200, one or more peripheral modules 250, a secondary electronic device 300, and a cloud computing device 400 all of which can be communicatively coupled, using either a wired or wireless connection. However, in some embodiments, the devices 100, 200, 250, 300, 400 shown in FIG. 1 do not always have to be connected to one another and may only establish a connection intermittently. Furthermore, in some embodiments, the solid pharmaceutical dosage form dispensing and biometric data acquisition device 100 may not connect to all the other devices 200, 250, 300, 400 shown in FIG. 1, but may only connect to one of the other devices 200, 250, 300, 400. For example, in some embodiments, the solid pharmaceutical dosage form delivery and biometric data acquisition device 100 may only connect to the secondary electronic device 300. In these embodiments, the secondary electronic device 300 may then connect to the cloud computing device 400, for example.

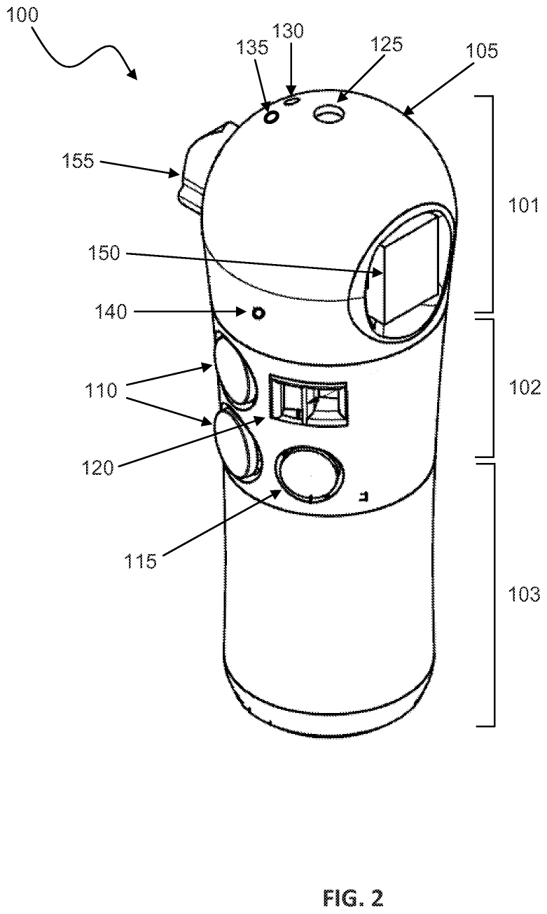

The solid pharmaceutical or other agent dosage form dispensing and biometric data acquisition device 100, the accessory module(s) 200, and the peripheral modules(s) 250 are discussed in more detail below in FIGS. 2-4. In the illustrated example, the secondary electronic device 300 can be a smartphone. However, other exemplary secondary electronic device(s) 300 can include, but are not limited to, a telephone, a laptop computer, a tablet computer, a personal digital assistant (PDA), a digital camera, a gaming device, a desktop computer, a fitness tracking device, a digital display device, a docking station, or a security terminal or station. The cloud computing device 400 may be implemented, for example, as one or more servers which may be communicatively coupled to the Internet, and which may be co-located or geographically distributed.

As illustrated in FIG. 2, the solid pharmaceutical or other agent dosage form dispensing and biometric data acquisition devices 100 of the present disclosure include a housing unit 105, which can be configured to contain a battery, a real time clock, and a processing device for operating a plurality of biometric sensors. The structure of the housing unit 105 can be generally configured to enable a user to grasp and operate the device without interfering with biometric data acquisition before, during, and/or after a solid pharmaceutical dosage form is dispensed. For example, in certain embodiments, the dispensing device can be cylindrical in shape, such that the device can be easily grasped and operated by one of the user's hand without difficulty, as illustrated in FIG. 2. Other similar handheld shapes or portable shapes and configurations can readily be ascertained by one of ordinary skill in the art based on the present disclosure.

In some embodiments, the solid pharmaceutical or other agent dosage form dispensing and biometric data acquisition devices 100 of the present disclosure can be constructed of three main portions or segments, a top portion 101, a middle portion 102, and a bottom portion 103, as illustrated in FIG. 2. In some aspects, the top segment 101, middle segment 102, and bottom segment103 are detachable to enable access to the internal contents of the device 100. The method or means for detachment and reattachment of the top 101, middle 102, and bottom 103 portions can include various methods known to one of ordinary skill in the art based on the present disclosure, including for example, bolts, screws, screw threads, snap locks, quick release button, alignment of the portions by indicator, pins, and the like. In some aspects, the top 101, middle 102, and bottom 103 portions are coupled together in a tamper-proof manner in order to restrict access to the internal contents of the device 100 (e.g., prevent access to the solid pharmaceutical agent dosage forms). For example, the top 101, middle 102, and bottom 103 portions can be sealed together with various tamper-proof adhesives and/or reinforced with tamper-proof screws or bolts. In some aspects, only two of the three top 101, middle 102, and bottom 103 portions are sealed in a tamper-proof manner, and in other aspects, all three of the top 101, middle 102, and bottom 103 portions are sealed in a tamper-proof manner.

Suitable materials that can be used to construct the housing unit 105 include, but are not limited to, various plastics and polymers materials, such as polystyrene (PS), polycarbonate (PC), acrylonitrile-butadiene-styrene (ABS), polybutylene terephthalate (PBTP), styrene acrylonitrile (SAN), polyamide (PA), polyoxymethylene (POM), polyphenylene oxide (PPO), PE, PP, PTFE and homopolymers and copolymers of these plastics. The plastics may also be used in a filled or fiber-reinforced form, and/or coupled to portions of metals or metal alloys, such as aluminum, titanium, steel, and combinations thereof. The materials used to construct the housing unit 105 can be surface-coated, for example with paints, varnishes or lacquers. The use of color plastics, for example colored with pigments, can also possible. In some embodiments, the housing unit 105 can be coated with substances that help to prevent contamination from microorganisms, bacteria, fungi, viruses, and the like. The coatings can be active solid pharmaceutical dosage forms that reduce the growth and/or survival of these harmful microorganisms (e.g., anti-bacterial substances), or the coatings can function passively to prevent contamination, for example, by preventing adherence of these microorganism to the housing unit 105 (e.g., wetting agents) or coated with agents to reduce moisture from penetrating the internal compartment.

In some embodiments, access to the internal contents of the device 100 can be restricted to authorized users, for example, authorized caregivers, pharmacists, physicians, and the like. In some aspects, the top 101, middle 102, and/or bottom 103 portions of the device 100 can be detached by an authorized user that has been identified and authenticated in order to obtain access to the contents of bottom portion 103 of the device 100. The authorized user can be identified and authenticated using one or more biometric scanners, such as a fingerprint scanner, as provided further herein.

In some aspects, once an authorized user has been authenticated, he or she can perform various operations that other users are restricted from performing. For example, an authorized user can reprogram the list of solid pharmaceutical dosage forms associated with a person's biometric identifier; an authorized user can add, subtract, or replace the number of solid pharmaceutical dosage forms contained within the device (e.g., refill a prescription); an authorized user can alter the dose of a solid pharmaceutical dosage form; and/or an authorized user can alter the dosing parameters (e.g., dispensing period) of a solid pharmaceutical agent dosage form. Restricting access to or maintaining control over dispensing the solid pharmaceutical agent dosage forms may be necessary if a user of the device 100 requires oversight, and/or to prevent misuse or abuse of the solid pharmaceutical dosage forms. In such situations, the device 100 provides increased safety and security, as compared to standard medication dispensing devices.

In other embodiments, once an authorized user has been authenticated and steps have been taken to fill or refill the solid pharmaceutical dosage forms in the device, a picture of the individual dosage form, or groups of dosage forms, to be dispensed may be demonstrated to the user. In some embodiments, the authorized/authenticated user (e.g., pharmacist or other healthcare professional) may show one or more pictures of the solid pharmaceutical dosage forms to the user who will be taking the dosage forms so that the user can verify if the correct dosage form(s) and the correct number of dosage forms have been dispensed. In some embodiments, the authorized/authenticated user can send one or more pictures of the dosage forms to the user's mobile/computing device and/or directly to the solid pharmaceutical dosage form dispensing and biometric data acquisition devices 100. In other embodiments, the pictures can be stored in the devices' respective memories so that when the corresponding dosage forms are dispensed, the user can view the picture to verify that the correct types and numbers of dosage forms have been dispensed prior to ingestion. If not, the user can provide feedback using the device and/or a mobile/computing device that indicates that types and/or numbers of dosage forms have not been accurately dispensed. This type of feedback can be accessed by an authenticated user and/or sent directly to an authenticated user for evaluation. This feature generally makes it easier for a user to determine if his or her medication has been accurately dispensed, without the need to memorize the types and quantities of the medication.

In some embodiments, the generally cylindrical shape of the housing unit 105 can provide sufficient structure for allowing the user to interface with various biosensors included in the device. For example, the middle portion 102 of the device 100 can include one or more galvanic skin response sensors 110 (FIG. 2). The galvanic skin response (GSR) sensors 110 of the present disclosure, also referred to as electrodermal response (EDR) sensors, psychogalvanic reflex (PGR) sensors, skin conductance response (SCR) sensors, or skin conductance level (SCL) sensors, generally measure electrical conductance of the skin, which varies depending, for example, on the state of sweat in the skin. Sweating is generally considered to be controlled by the sympathetic nervous system; therefore, electrical skin conductance can provide psychological and/or physiologic biometric data about the user. In general, if the sympathetic branch of the autonomic nervous system is highly aroused, then sweat gland activity also increases, which in turn increases skin conductance. In this way, skin conductance can be used as a biometric measurement of emotional and sympathetic responses, which can be used to evaluate, for example, the efficacy and/or side effects caused by various solid pharmaceutical or other agent dosage forms before, during, or after dispensing the solid pharmaceutical dosage forms.

In other embodiments, the housing unit 105 can provide sufficient structure for incorporating one or more temperature sensors (FIG. 2). For example, the middle portion 102 of the device 100 can include one or more fingertip temperature sensors 115 to enable the acquisition of the temperature of a user's skin before, during, or after dispensing a solid pharmaceutical dosage form. Typically, the temperature at the surface of a subject's skin changes according to blood circulation through the body tissue. The small blood vessels crossing through the tissue are surrounded by fibers of smooth muscle, which are controlled by the sympathetic nervous system. In a state of increased exertion, excitement and stress, these muscle fibers contract, causing a stenosis of vasculature. This can lead to a reduction of skin temperature, because blood circulation through the tissue is reduced. In contrast, in a state of relaxation, the musculature is likely to relax, causing the vasculature to expand. Hence, skin temperature rises. Mental stress often leads to a lower peripheral perfusion and a decrease of skin temperature at the hands, caused by increased activity of the sympathetic nervous system. In this way, temperature of the skin at a user's fingertip can be used to as a biometric measurement for evaluating, for example, the efficacy and/or side effects caused by various solid pharmaceutical dosage forms before, during, or after dispensing a solid pharmaceutical dosage form.

In some embodiments, the housing unit 105 can provide sufficient structure for incorporating one or more ambient temperature sensors for measuring the air temperature immediately surrounding the device, thus reflecting changes in the environmental conditions. In some embodiments, an ambient temperature sensor can be integrated with the galvanic skin response sensors and/or the fingertip temperature sensors in order to account for alterations in the environmental conditions. The integration of the various temperature sensors can provide more accurate temperature measurements of the subject for evaluating, for example, the efficacy and/or side effects caused by various pharmaceutical agents before, during, or after delivery of the pharmaceutical agents.

In other embodiments, a fingertip sensor can be included in the devices of the present disclosure to measure a user's heart rate. For example, a fingertip heart rate sensor unit can include an infrared light-emitting-diode (IR LED) and a photo diode, such that a user's fingertip can be placed over the sensor unit. The IR LED can transmit an infrared light into the fingertip, a part of which can be reflected back from the blood inside the finger arteries. The photo diode then senses the portion of the light that is reflected back. The intensity of reflected light depends upon the blood volume inside the fingertip, which varies every time the heart beats in accordance with changes in the amount of reflected infrared light detected by the photo diode. Other similar methods of detecting heart rate using a fingertip sensor can readily be ascertained by one of ordinary skill in the art based on the present disclosure. Monitoring a user's heart rate can be an important biometric measurement for evaluating, for example, the efficacy and/or side effects caused by various solid pharmaceutical dosage forms before, during, and/or after dispensing a solid pharmaceutical dosage form.

In some embodiments, the housing unit 105 can provide sufficient structure for incorporating one or more pulse oximeters 120 (FIG. 2). For example, a pulse oximeter 120 can be used to measure the oxygen level (or oxygen saturation) in a subject's blood. Typically, the pulse oximeter 120 can be placed on a thin part of a subject's body, usually a fingertip, and two wavelengths of light are passed through the fingertip to a photodetector. The photodetector measures the changing absorbance at each of the wavelengths, allowing it to determine the absorbance due to the pulsing arterial blood alone. The pulse oximeter 120 can be used to assess a user's blood oxygenation levels and determining the effectiveness of, or need for, supplemental oxygen. The pulse oximeter 120 can also be used as a biometric measurement for evaluating, for example, the efficacy and/or side effects caused by various solid pharmaceutical dosage forms before, during, or after dispensing a solid pharmaceutical dosage form. The pulse oximeter 120 can also be used to determine noninvasively a subject's hemoglobin level within 1-2 minutes without requiring any further equipment.

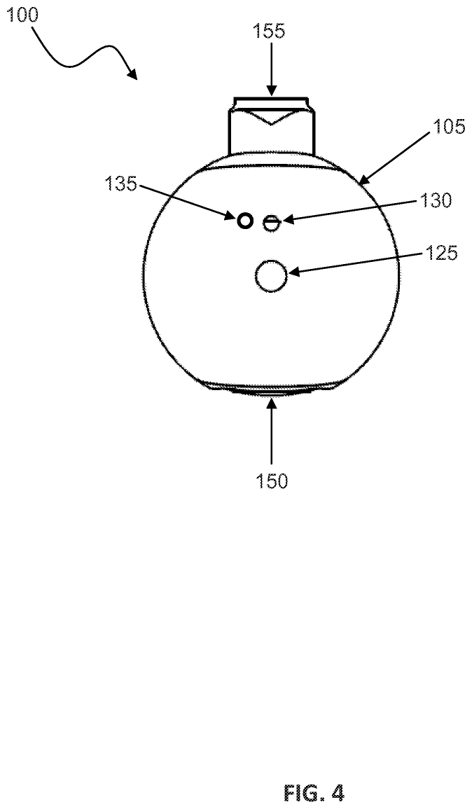

Other embodiments can include one or more scanners associated with the device which authenticate and/or verify the identity of a user or caregiver that will be dispensing a solid pharmaceutical dosage form. In some embodiments, the top portion 101 of the device 100 includes an image acquisition device 130 (FIGS. 2 and 4). The image acquisition device 130 generally comprises a lens, an image sensor, and signal wires which operatively connect the image acquisition device 130 to the processor in the device. In some embodiments, the image acquisition device can be a digital camera. The image acquisition device 130 can be centrally located on the top portion 101 of the device 100, and be electrically coupled to the processor of the device.

In one manner of operation, the image acquisition device 130 can take a digital image and/or a series of digital images (e.g., a digital video) before, during, and/or after dispensing a solid pharmaceutical dosage form. In some embodiments, the image sensor can detect a user's pupils and capture one or more images of the user's pupils before, during, and/or after dispensing a solid pharmaceutical dosage form in order to assess the efficacy and/or side effects caused by the solid pharmaceutical dosage form. In some embodiments, the image acquisition device 130 can be used to assess the color of a user's eye, including but not limited to, the color of a user's sclera. For example, certain conditions can cause a subject's eyes to appear yellow, which can indicate dysfunction in one or more bodily organs such as the liver, gallbladder, or pancreas. Yellowing of the sclera can be used to diagnose various conditions of a user, including, but not limited to, alcohol abuse, hepatitis (A, B, C, D, and E), liver cancer, liver infection, and non-alcoholic fatty liver disease.

In addition, an image acquisition device 130 can be configured to capture a digital image and/or a series of digital images that can be transferred to an auxiliary electronic device and viewed by a caregiver for diagnostic purposes. For example, a pharmaceutical agent delivery and biometric data acquisition device 100 can have an activation button functionally coupled to the image acquisition device 130 to enable a user to engage the activation button and capture a digital image or video of, for example, information pertaining to the pharmaceutical agent (e.g., dose, lot number, etc.) or a physical manifestation of a disease condition located on the subject (e.g., wound, laceration, allergic reaction, insect bite, swollen glands, etc).

In some embodiments, the image acquisition device 130 can be configured to take a picture of a subject's retina to evaluate the vascularization of the retina and/or whether the subject has a retinal vascular occlusion. A retinal vascular occlusion occurs when one of the veins or arteries carrying blood to or from the retina becomes blocked or contains a blood clot. The blockage could occur in the main vein or main artery. Blockages could also occur in the branch of veins and arteries throughout the retina. A blockage in the vein or artery of the retina can cause blood or other fluids to build up and inhibit the retina's ability to filter light properly. When light is blocked or fluids are present, sudden loss of vision can occur. The presence of a retinal vascular occlusion or blockage can be a predictor of an increased likelihood that the subject will experience a stroke.

The pharmaceutical agent delivery and biometric data acquisition device 100 can also be equipped with a microphone that may or may not be functionally coupled to the image acquisition device 130 to facilitate real-time and/or recorded audio and/or video communication with a caregiver for diagnostic purposes (e.g., videoconferencing).

In some embodiments, the image acquisition device 130 faces the same direction as that of a centrally located airflow port 125, located on the top portion 101 of the device 100. The airflow port 125 can be coupled to an airflow port accessory module, such as, for example, a mouthpiece or a mask (e.g., oxygen mask). When a user's mouth engages the airflow port accessory module, which is coupled to the airflow port 125, the user's eyes will be facing the lens of the image acquisition device 130.

In some embodiments, airflow port 125 facilitates the inhalation and exhalation of air from a user to the device 100, thus enabling the velocity, depth and composition of a subject's breath to be measured. These pulmonary biometrics can be important metrics for evaluating the health of the subject and/or the efficacy of the solid pharmaceutical dosage form. For example, the device of the present disclosure can include one or more air pressure sensors which measure air pressure, often stated in terms of force per unit area. A pressure sensor typically acts as a transducer by generating an electrical or digital signal as a function of the pressure imposed. Sensors can be used to measure variables such as air flow, speed, and altitude. Air pressure sensors can alternatively be called pressure transducers, pressure transmitters, pressure senders, pressure indicators, piezometers and manometers, among other names, as would be appreciated by one of ordinary skill in the art based on the present disclosure.

In some embodiments, air pressure sensors can be coupled with one or more sensors designed to assess the chemical and/or gaseous composition of a user's breath. For example, the device of the present disclosure can include one or more sensors to detect carbon dioxide levels. A carbon dioxide sensor or CO₂ sensor typically includes infrared gas sensors (e.g., NDIR sensors) and chemical gas sensors, which can help assess the function of a subject's lungs. NDIR sensors are typically spectroscopic sensors used to detect CO₂ by its characteristic absorption. The key components include an infrared source, an interference (wavelength) filter, and an infrared detector. In some embodiments, a user breathes air through a mask or mouthpiece coupled to the airflow port 125, and the sensor measures the absorption of the characteristic wavelength of light. CO₂ sensors can also be functionally coupled with one or more air pressure sensors described above to capture a user's biometric data pertaining to both CO₂ levels and respiration rate, key biometrics used to evaluate a subject's health and disease state. Air pressure sensors and CO₂ sensors can also be used to assess, for example, the efficacy and/or side effects caused by various solid pharmaceutical dosage forms before, during, or after dispensing the solid pharmaceutical dosage form. In other embodiments, sensors can be used to detect odors in a subject's breath, including an ammonia-like odor, which can be indicative of kidney failure, and/or a fruity odor, which can be indicative of ketoacidosis/diabetes.

Other sensors can also be included in the devices of the present disclosure, as would be readily appreciated by one of ordinary skill in the art based on the present disclosure. For example, the devices of the present disclosure can include global positioning system (GPS) sensors, chemical sensors, thermal sensors, magnetic sensors, radiation sensors, proximity sensors, acoustic sensors, vibration sensors, acceleration sensors, moisture sensors, and the like. In some embodiments, the device can be equipped with a sensor or monitor capable of measuring a subject's blood glucose levels, as well as determining if the subject's blood glucose levels are within a certain range.

In other embodiments, a thermal imaging sensor can be included in the devices of the present disclosure to facilitate user authentication and/or as a biometric sensor. A thermal imaging sensor can be integrated with the image acquisition device to facilitate the scanning and processing of a thermal image of one or more portions of a subject's face and/or the subject's entire body. In some embodiments, the thermal imaging sensor can be used to evaluate whether the subject has a medical condition (e.g., fever) that may require immediate attention. In such embodiments, the device can be configured to send an alert message to the subject to seek immediate medical attention.

In some embodiments, a user (e.g., authenticated user, healthcare provider or associate of the authenticated user or other authorized personnel) can set one or more alarms using the device, such as one or more medication alarms, which can present a stimulus to the user with or without an accompanying text-based message to, for example, take one or more doses of one or more pharmaceutical or biological agents. The alarm can be a visual (e.g., flashing light) and/or auditory (e.g., ringing bell sound) stimulus that can be emitted from the device. The alarm can also be pushed to another device, such as a mobile phone or computing device. In some embodiments, the alarm can take the form of an email, text message, a message from a third party mobile phone application and the like. Similarly, a user can set one or more biometric alarms, which can present a similar stimulus to the user to, for example, obtain and record one or more biometrics using the device.

The device 100 can also include one or more visual indicators that emit at least one light signal, such as a green, red, or white colored light signal. In some embodiments, the visual indicator can be an LED 135 located in the top portion 101 of the device 100, adjacent to the image acquisition device 130 (FIGS. 2 and 4). LED 135 can be operatively coupled to the image acquisition device 130 to provide a visual cue to the user indicating, for example, when the image acquisition device has been activated, deactivated, and/or is in the process of capturing images. In other embodiments, the visual indicator can be an LED 140 located on the side portion of the top portion 101 of the device 100, adjacent to one or more biometric sensors (FIG. 2). These and other visual indicators can be used to communicate instructions to the user, such as indicating whether the user has been authenticated, or when to dispense a solid pharmaceutical dosage form. These and other visual indicators can also be used to facilitate the acquisition of biometric data from the user, such as emitting a flash of light to dilate a user's pupils. Changes in a user's pupil size or pupil dilation can be an important biometric measurement indicating, for example, the efficacy and/or side effects caused by solid pharmaceutical dosage form.

In accordance with these embodiments, negative visual indicators can then be used to adjust, change or eliminate use of the agent for the user. Additionally, the device can be configured to send instructions to a user to activate an eye tracking program that uses visual stimulation, such as pulses of light, to assess various neurological problems, including brain diseases and brain injuries (e.g., concussions). Eye tracking technology and testing protocols are well established and can obtain hundreds of data points during, for example, a 30-second test facilitated by the video recording capability of the image acquisition device 130.

Other embodiments can include one or more scanners on the device which authenticate and/or verify the identity of a user or caregiver that will be dispensing a solid pharmaceutical dosage form to a subject. In some embodiments, images captured using the image acquisition device 130 can be used for retinal scanning and/or facial recognition to prevent unauthorized users from dispensing a solid pharmaceutical dosage form and/or tampering with the device. In other embodiments, the device of the present disclosure includes a fingerprint scanner 150 to prevent unauthorized users from dispensing a solid pharmaceutical dosage form and/or tampering with the device, as explained above (FIGS. 2-4).

For example, the device 100 of the present disclosure can store in its memory a plurality of distinct user fingerprints (e.g., biometric identifiers), and the device 100 can be programmed to correlate a particular fingerprint with certain device settings for a particular user. In this way, the device 100 can be used by more than one user, for example, a family of users, without the need for multiple devices for each person or solid pharmaceutical dosage form being dispensed. In other embodiments, the device 100 can be configured to be accessed only by an authorized user such as a nurse, parent or other caregiver, and the nurse, parent or caregiver's fingerprint or other biometric identifier can be used to access the patient's settings on the device, as the patient may need to be restricted from using the device or the patient may not be capable of using the device without supervision (e.g., a child or elderly person). The fingerprint scanner 150 can also be used in conjunction with a lockout mechanism in which the device will be “locked out” or inactive for a given operation of a particular delivery program if the user's fingerprint is not recognized.

The device 100 also includes memory in electrical communication with the processor of the device and configured to facilitate the acquisition and storage of biometric data acquired using various biometric sensors from one or more users. Biometric data can include, but is not limited to, images, air flow rates, air composition, fingerprints, oxygen levels, carbon dioxide levels, skin electrical conductance measurements, time, temperature, heat, user identification, dosages, usage rates, medication batch numbers, bar codes, and any other biometric data that can be captured using the various biometric sensors of the present disclosure. User biometric data can be stored on the memory and uploaded/downloaded wirelessly to a variety of other memory storage and data processing devices, including but not limited to, cell phones, smart phones, watches, computers, laptops, tablets, servers, and the like. User biometric data can also be stored on the memory and uploaded/downloaded via a wire or cable to a variety of other memory storage and data processing devices, including but not limited to, cell phones, smart phones, watches, computers, laptops, tablets, servers, and the like. In such embodiments, the device can have one or more data transfer ports. The ability to acquire and store a subject's biometric data over time provides physicians with more accurate diagnostic and biometric data with which to evaluate the subject, and allows for more general patient trends to be analyzed with relation to, for example, a specific disease indication.

Certain embodiments disclosed herein concern, a solid pharmaceutical dosage dispensing and biometric data acquisition device having various mechanisms for dispensing one or more solid pharmaceutical dosage forms to a user. In one embodiment, the bottom portion 103 of device 100 comprises a loading cartridge that can be accessed by a user or caregiver, as described above, in order to load one or more solid pharmaceutical dosage forms into the device 100. The loading cartridge can be comprised of a plurality of storage chambers that are configured to house one or more solid pharmaceutical dosage forms prior to being dispensed to a user. In some embodiments, the storage chambers are rotatable (e.g., mounted on rotatable discs) such that the vertical alignment of a storage chamber containing one or more solid pharmaceutical dosage forms with one or more vacant storage chambers facilitates the release or ejection of the solid pharmaceutical dosage forms from the device 100.

In some embodiments, the device 100 contains a motion sensor circumferentially located in the bottom portion 103 of the device 100 to detect the release or ejection of one or more solid pharmaceutical dosage forms. Motion sensors can be used to detect a change in the position of a solid pharmaceutical dosage form as it exits the device 100. Motion detection can be achieved by mechanical and/or electronic methods, including but not limited to, infrared (passive and active sensors), optics (video and camera systems), radio frequency energy (radar, microwave and tomographic motion detection), sound (microphones and acoustic sensors), vibration (triboelectric, seismic, and inertia-switch sensors), and magnetism (magnetic sensors and magnetometers).

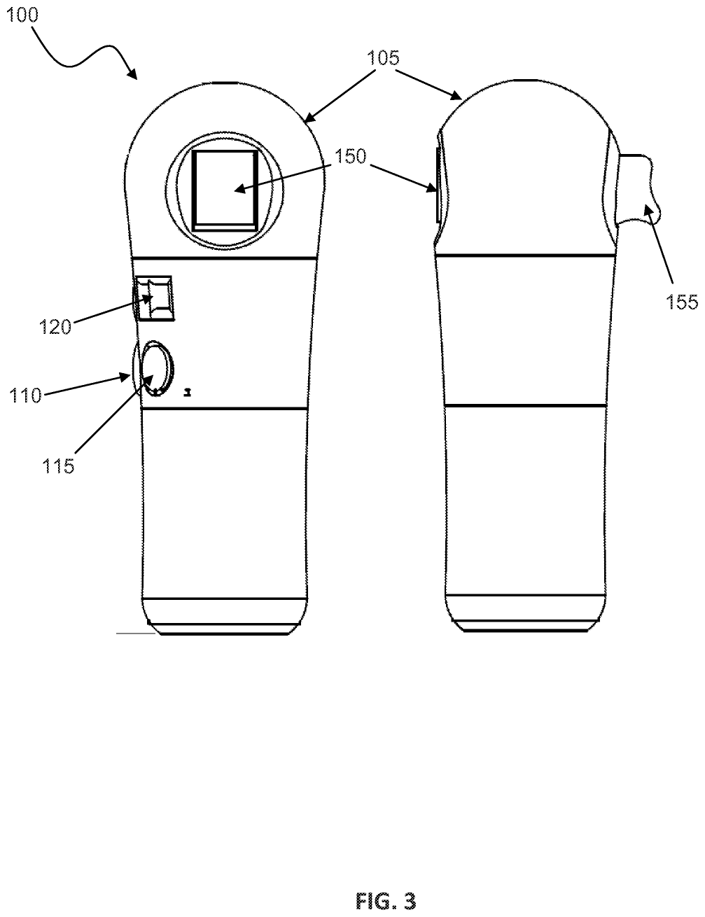

As part of one mechanism for dispensing one or more solid pharmaceutical dosage forms, the device 100 can include, but is not limited to, a prime mover operably coupled to the loading cartridge and a dispensing element 155. The dispensing element 155 can be located in the top portion 101 of the device 100, as shown in FIG. 3, such that the user can engage the dispensing element 155 conveniently and easily while the device 100 is held in the user's hand (e.g., with a trigger like motion of the user's index finger). In one manner of operation, a user's engagement with the dispensing element 155 actuates the prime mover, which in turn facilitates the release or ejection of one or more solid pharmaceutical dosage forms from the loading cartridge. The release of one or more solid pharmaceutical dosage forms generally constitutes a single dose, and the time at which the one or more solid pharmaceutical dosage forms is dispensed to a user can be recorded and stored in the memory of the device 100, as explained herein.

In some embodiments, a user's engagement with the dispensing element 155 will not lead to the release of one or more solid pharmaceutical dosage forms if the user's biometric identifier has not been authenticated (e.g., the user is not authorized to dispense that particular solid pharmaceutical dosage form or is not authorized to use the device). In some embodiments, the dispensing element 155 can be coupled to a scanner, such as the fingerprint scanner 150, and the the dispensing element 155 will be in a “locked” position until a user has been authenticated, at which time the dispensing element 155 will be in an “unlocked” position and can actuate the prime mover to release one or more solid pharmaceutical dosage forms. In some embodiments, once the dispensing of one or more solid pharmaceutical dosage forms is sensed by the motion sensor, the dispensing element 155 will be placed in a “locked” position in order to prevent a user from dispensing more than one dose of a solid pharmaceutical dosage form.

Other mechanisms to facilitate the release and/or ejection of the one or more solid pharmaceutical dosage forms can also be used with the device 100, as would be readily apparent to one of skill in the art based on the present disclosure.

Embodiments disclosed herein can also include one or more accessory module interfaces on a device that facilitates the functional coupling of one or more accessory modules 200 to the device. Examples of accessory modules 200 include, but are not limited to, an injectable syringe, an injectable needle, an inhaler, an inhaler canister, a syrup dispenser, a spray device, a nebulizer, a misting device, an inhalation mask, and the like. The accessory module interfaces allow for one or more accessory modules 200 to be coupled to the device. In some embodiments, the device will also include a mechanism for monitoring the amount or dosage of a solid pharmaceutical dosage form dispensed to a subject. For example, the device can include a radio-frequency identification (RFID) reader, which can be used to assess the batch, date, amount and source of a particular solid pharmaceutical dosage form.

In some embodiments, the device includes an accessory module 200 that acts as a storage container for various pharmaceutical and biological agents in various physical forms (e.g., mists, sprays, liquids, solid dosage forms, syrups and the like). In one manner of use, for example, the storage container can be inserted into the device, and the device records the presence of the storage container. The device can be equipped with sensors to not only detect the presence or absence of the storage container, but also the weight of the storage container. When a user manually engages with an interface on the device to eject the storage container, the device can record the time that the storage container was ejected. The user can then open or in some way manually access the contents of the storage container in order to administer one or more pharmaceutical or biological agents. For example, the user can remove from the storage container an eye dropper and administer a specific number of drops to his or her eyes. After administering the one or more pharmaceutical or biological agents, the user can reinsert the storage container in the device and this time can be recorded as well (e.g., to determine whether the user is complying with a predetermined treatment plan). The weight sensors in the device can then record whether the weight of the container has changed, and if so, this can be an indication as to whether a predetermined dose of a pharmaceutical or biological agent was administered.

In some embodiments, peripheral accessory modules or peripheral modules can be functionally coupled to the device of the present disclosure via peripheral module interfaces rather than accessory module interfaces. Typically, a peripheral module requires its own power source separate from the device, which can preclude the peripheral module from being coupled to the device via an accessory module interface. The peripheral accessory interface can be a port, including any electronic data transfer port, such as a USB port, a firewire port, and the like. Peripheral modules can include, for example, blood pressure monitors, blood glucose monitors, CPAP machines, and/or electrocardiogram machines, as well as peripheral modules for providing additional power to the device, such as a battery or a battery charging device, and devices that enable the use of Bluetooth and WiFi compatibility. Just as with accessory modules, some peripheral modules can be functionally coupled to the processor of the device of the present disclosure to facilitate the dispensing of a solid pharmaceutical dosage form and/or the acquisition of biometric data from a user.

In some embodiments, the device can be coupled to a CPAP machine (Continuous Positive Airway Pressure) or a baby monitor (e.g., monitors used to assess Sudden Infant Death Syndrome, or SIDS), or other such medical monitoring peripheral devices. The device can be used to acquire further biometric data that is not possible using the medical monitoring peripheral device, and/or the device can be used to integrate the biometric data acquired using the medical monitoring peripheral device. In other embodiments, the device can be coupled to a motor vehicle, such that operation of the motor vehicle (e.g., starting a car) by the subject will only be allowed if certain biometric parameters are met. This feature can help prevent a subject who is taking various pharmaceutical and biological agents from operating a motor vehicle while impaired.

In some embodiments, a peripheral module can be a secondary electronic device, such as a docking station. The docking station can be used to charge the device, and can include various other accessory ports, such as an Ethernet port and/or a communication port to support a telephone landline. Additionally, the docking station can be configured to sterilize the device between uses and/or between uses by multiple users to minimize and/or prevent bacterial, fungal, and viral contamination. For example, the docking station can be configured to contain one or more sources of UV light to reduce contamination when the device is housed in the docking station. The docking station can also be configured to combine the sterilization power of UV light, purifying hydroxyl and/or activated oxygen radicals, and photo-ionization to purify the internal and external components of the device. To facilitate this sterilization process, the docking station can be equipped with various air flow mechanisms, which assist with both the activation and circulation of the hydroxyl and oxygen radicals through the device. These and other sterilization mechanisms can be included in the docking station, as would be readily recognized by one of ordinary skill in the art based on the present disclosure.

Various solid pharmaceutical dosage forms of an agent can be dispensed using the devices, systems, and methods of the present disclosure, including both prescription and non-prescription (e.g., “over the counter” or OTC) solid dosage agent forms. Solid pharmaceutical agent dosage forms can include vitamins, minerals, nutraceuticals, supplements and various combinations of these. Solid pharmaceutical agent dosage forms can include active pharmaceutical ingredients (APIs) that contain both solid and liquid components. A liquid component can be, for example, fish oil or other liquid form of a pharmaceutical or biological agent. Solid pharmaceutical dosage forms can be one or more of pill, capsule, gel capsule, gummy, timed-release capsule, slow-dissolving capsule, tablet, caplet, patch, strip, and thin-film strip, mini-tablets, granules, beads, pellets, multiparticulates, spheroids, or combinations thereof.

In some embodiments, a solid pharmaceutical agent dosage form can be in the form of a strip or a thin-film strip contained within and dispensed by the solid pharmaceutical dosage dispensing and biometric data acquisition device. Generally, thin-film strips are designed to deliver one or more doses of a solid pharmaceutical agent dosage form to a subject using a dissolving film or oral strip to administer a pharmaceutical agent via absorption in the mouth (buccally or sublingually) and/or via the small intestines (enterically). A film or thin-film strip can be prepared using hydrophilic polymers that rapidly dissolve on the tongue or buccal cavity, delivering the pharmaceutical agent to the systemic circulation via dissolution when contact with liquid is made. A film or thin-film strip can also include pH-sensitive microparticles and or mucoadhesive polymers that facilitate the release of the solid pharmaceutical dosage form in various portions of a subject's intestinal tract (e.g., small intestine, gastric cavity, etc.). In some embodiments, a film or thin-film strip comprising a solid pharmaceutical dosage form can also include a copolymer of methacrylic acid or acrylic acid, such as a Eudragit-style copolymer, a pluronic polymer, a chitosan, a chitosan derivative, a surfactant, a sugar, a buffering salt, or a combination thereof.

In some embodiments, a film or thin-film strip comprising a solid pharmaceutical dosage form can be sealed in a sterile package with perforations demarcating each dose to facilitate the removal of a dose from the solid pharmaceutical agent dosage dispensing and biometric data acquisition device by a user. In other embodiments, removal of a dose of the pharmaceutical agent from the device, such as by tearing along the perforation, can be sensed and recorded in the memory of the device in order to monitor the administration of the pharmaceutical agent to the user.

In other embodiments, a solid pharmaceutical agent dosage form can be in the form of a patch contained within, and dispensed by, the solid pharmaceutical agent dosage dispensing and biometric data acquisition device. For example, the solid pharmaceutical dosage agent dispensing and biometric data acquisition device can include various patches that can be dispensed to an authorized user so that a pharmaceutical agent can be delivered through the skin without the use of a needle or a syringe (needleless). In some embodiments, the patch can include protein-based pharmaceutical agents that are between about 500 daltons (da) and about 150 kilodaltons (kDa), such as a vaccine or a biologic agent. The protein-based pharmaceutical agents can be in the form of nanofibrils and/or nanofibers that adhere to a surface of the patch and are stable at room temperature, as well as lower temperatures (standard refrigerated temperatures), for 20 to about 30 weeks. The patch enhances the surface area in which the pharmaceutical agent is in contact with a subject's skin, thus allowing for efficient delivery of the pharmaceutical agent at much lower doses than the conventional injection of liquid-based formulations.

In addition to dispensing patches and/or strips comprising protein-based pharmaceutical agents, the solid pharmaceutical dosage dispensing and biometric data acquisition device can also be configured to dispense various other types of medication on patches and strips to an authorized user, such as small molecule drugs, and/or a combination of protein-based and small molecule pharmaceutical agents, as would be readily recognized by one or ordinary skill based on the present disclosure.

Solid pharmaceutical dosage agent forms that can be dispensed using the devices, systems, and methods of the present disclosure can include, but are not limited to, those approved by the U.S. Food and Drug Administration, such as, for example, albuterol, albuterol sulfate, atropine sulfate, beclomethasone dipropionate, bitolterol mesylate, budesonide, formoterol fumarate, cromolyn sodium, desflurane, dexamethasone sodium phosphate, dornase alfa, enflurane, epinephrine, ergotamine tartrate, flunisolide, fluticasone propionate, fomoterol fumarate, halothane, iloprost, insulin, ipratropium bromide, isoetharine hydrochloride, isoflurane, isoproterenol hydrochloride, levalbuterol hydrochloride, metaproterenol sulfate, methacholine chloride, mometasone furoate, nedocromil sodium, nicotine, nitric oxide, pentamidine isethionate, pentetate calcium trisodium, pentetate zinc trisodium, pirbuterol acetate, ribavirin, salmeterol xinafoate, sevoflurane, tetrahydrocannabinol, tiotropium bromide monohydrate, tobramycin, trimcinolone acetonide, zanamivir, and combinations and derivatives thereof.

Solid pharmaceutical dosage forms that can be dispensed using the devices, systems, and methods of the present disclosure include, but are not limited to, those solid pharmaceutical dosage forms that have not yet been approved by the U.S. Food and Drug Administration, such as, for example, 13-cis-retinoic acid, 2-pentenylpenicillin, L-alphaacetylmethadol, S-adenosylmethionine, acebutolol, aceclofenac, acetaminophen, acetaphenazine, acetophenazine, ademetionine, adinazolam, adrafinil, ahnotriptan, albuterol, albuterol, albuterol sulfate, alfentanil, alfentanil HCl, alizapride, allylprodine, alminoprofen, almotriptan, alperopride, alphaprodine, alpidem, alseroxion, amantadine, ambrisentan, amesergide, amfenac, aminopropylon, amiodarone HCl, amisulpride, amitriptyline, amixetrine, amlodipine, amoxapine, amoxicillin, amperozide, amphenidone, amphetamine, ampicillin, amylpenicillin, andropinirole, anileridine, apazone, apomorphine, apomorphinediacetate, atenolol, atropine sulfate, azacyclonol, azasetron, azatadine, azidocillin, bacille Calmette-Guerin, baclofen, beclomethasone dipropionate, benactyzine, benmoxine, benoxaprofen, benperidol, benserazide, benzpiperylon, benzquinamide, benztropine, benzydramine, benzylmorphine, benzylpenicillin, bezitramide, binedaline, biperiden, bitolterol, bitolterol mesylate, brofaromine, bromfenac, bromisovalum, bromocriptine, bromopride, bromperidol, brompheniramine, brucine, buclizine, budesonide, budesonide; formoterol fumarate, budipine, bufexamac, buprenorphine, bupropion, buramate, buspirone, butaclamol, butaperazine, butorphanol, butriptyline, cabergoline, caffeine, calcium-N-carboamoylaspartate, cannabinoids, Cannabis, Cannabis oil, captodiamine, capuride, carbamazepine, carbcloral, carbenicillin, carbidopa, carbiphene, carbromal, carfecillin, carindacillin, caroxazone, carphenazine, carpipramine, carprofen, cefazolin, cefinetazole, cefinetazole, cefoxitin, cephacetrile, cephalexin, cephaloglycin, cephaloridine, cephalosporin C, cephalosporins, cephalotin, cephamycin A, cephamycin B, cephamycin C, cephamycins, cepharin, cephradine, cericlamine, cetrizine, chloralbetaine, chlordiazepoxide, chlorobutinpenicillin, chlorpheniramine, chlorpromazine, chlorprothixene, choline, cialis, cilazaprol, cilostazol, cinchophen, cinmetacin, cinnarizine, cipramadol, citalopram, clebopride, clemastine, clobenzepam, clocapramine, clomacran, clometacin, clometocillin, clomipramine, clonidine, clonitazene, clonixin, clopenthixol, clopriac, clospirazine, clothiapine, clovoxamine, cloxacillin, clozapine, codeine, cotinine, cromolyn sodium, cyamemazine, cyclacillin, cyclizine, cyclobenzaprine, cyclosporin A, cyproheptadine, deprenyl, desflurane, desipramine, dexamethasone sodium phosphate, dexfenfluramine, dexmedetomidine, dextroamphetamine, dextromoramide, dextropropoxyphene, diamorphine, diazepam, diclofenac, dicloxacillin, dihydrocodeine, dihydroergokryptine, dihydroergotamine, diltiazem, diphenhydramine, diphenicillin, diphenidol, diphenoxylate, dipipanone, disulfiram, dolasetronmethanesulfonate, domeridone, dornase alfa, dosulepin, doxepin, doxorubicin, doxylamine, dronabinol, droperidol, droprenilamin HCl, duloxetine, eletriptan, eliprodil, enalapril, enciprazine, enflurane, entacapone, entonox, ephedrine, epinephrine, eptastigmine, ergolinepramipexole, ergotamine, ergotamine tartrate, etamiphyllin, etaqualone, ethambutol, ethoheptazine, etodolac, famotidine, fenfluramine, fentanyl, fexofenadine, fientanyl, flesinoxan, fluconazole, flunisolide, fluoxetine, flupenthixol, fluphenazine, flupirtine, flurazepam, fluspirilene, fluticasone propionate, fluvoxamine, formoterol fumarate, frovatriptan, gabapentin, galanthamine, gepirone, ghrelin, glutathione, granisetron, haloperidol, halothane, heliox, heptylpenicillin, hetacillin, hydromorphone, hydroxyzine, hyoscine, ibuprofen, idazoxan, iloprost, imipramine, indoprofen, insulin (recombinant human), ipratropium bromide, iproniazid, ipsapiraone, isocarboxazid, isoetharine hydrochloride, isoflurane, isometheptene, isoniazid, rifampin, pyrazinamide, ethambutol, isoproterenol, isoproterenol hydrochloride, isoproterenol bitartrate, isosorbide dinitrate, ketamine, ketoprofen, ketorolac, ketotifen, kitanserin, lazabemide, leptin, lesopitron, levalbuterol hydrochloride, levodopa, levorphanol, lidocaine, lisinopril, lisuride, lofentanil, lofepramine, lomustine, loprazolam, loratidine, lorezepam, loxapine, maprotoline, mazindol, mazipredone, meclofenamate, mecloqualone, medetomidine, medifoxamine, melperone, memantine, menthol, meperidine, meperidine HCl, meptazinol, mesoridazine, metampicillin, metaproterenol, metaproterenol sulfate, methacholine chloride, methadone, methaqualone, methicillin, methprylon, methsuximide, methyphenidate, methyprylon, methysergide, metoclopramide, metofenazate, metomidate, metopimazine, metopon, metoprolol, metralindole, mianserin, midazolam, milnacipran, minaprine, mirtazapine, moclobemide, mofegiline, molindrone, mometasone furoate, morphine, nabilone, nadolol, nafcillin, nalbuphine, nalmefene, nalorphine, naloxone, naltrexone, naratriptan, nedocromil, sodium, nefazodone, nefopam, nicergoline, nicotine, nicotine, nifedipine, nisoxetine, nitrous oxide, nitroglycerin, nomifensine, nortriptyline, obestatin, olanzapine, omoconazole, ondansetron, orphenadrine, oxprenolol, oxycodone, palonosetron, papaveretum, papaverine, paroxetine, pemoline, penfluridol, penicillin N, penicillin O, penicillin S, penicillin V, pentamidine isethionate, pentazocine, pentetate, calcium trisodium, pentetate, zinc trisodium, pentobarbital, peptides, pergolike, pericyazine, perphenazine, pethidine, phenazocine, pheneizine, phenobarbital, phentermine, phentolamine, phenyhydrazine, phosphodiesterase-5, pilocarpine, pimozide, pipamerone, piperacetazine, pipotiazine, pirbuterol acetate, pirbuterolnaloxone, piroxicam, pirprofen, pizotifen, pizotyline, polyeptides, polypeptide YY, pramipexole, prentoxapylline, procaine, procaterol HCl, prochlorperazine, procyclidine, promazine, promethazine, propacetamol, propanolol, propentofylline, propofol, propoxyphene, propranolol, proteins, protriptyline, quetiapine, quinine, rasagiline, reboxetine, remacemide, remifentanil, remoxipride, retinol, ribavirin, rimonabant, risperidone, ritanserin, ritodrine, rizatriptan, roxindole, salicylate, salmeterol xinafoate, salmetrol, scopolamine, selegiline, sertindole, sertraline, sevoflurane, sibutramine, sildenafil, spheramine, spiperone, sufentanil, sulpiride, sumatriptan, tandospirone, terbutaline, terguride, testosterone, testosterone acetate, estosterone enanthate, testosterone proprionate, tetrahydrocannabinol, thioridazine, thiothixene, tiagabine, tianeptine, timolol, tiotropium bromide monohydrate, tizanidine, tobramycin, tofenacin, tolcapone, tolfenamate, tolfenamicacid, topiramate, tramadol, tranylcypromine, trazadone, triamcinolone acetonide, triethylperazine, trifluoperazine, trifluperidol, triflupromazine, trihexyphenidyl, trimeprazine, trimethobenzamide, trimipramine, tropisetron, tryptophan, valproicacid, vardenafil, venlafaxine, verapamil, vigabatrin, viloxazine, yohimbine, zafirlukast, zalospirone, zanamivir, zileuton, ziprasidone, zolmitriptan, zolpidem, zopiclone, zotepine, zuclopenthixol, and combinations and derivatives thereof.

In some embodiments, the pharmaceutical agent delivery and biometric data acquisition devices of the present disclosure can be used to administer unapproved drugs, pre-approved drugs, and/or drugs subject to clinical trials. For example, the devices can be used to assess the efficacy of various pharmaceutical and biological agents that are being evaluated in the context of a clinical trial or other test. Test subjects can use the device in conjunction with clinical research being conducted to evaluate a drug's ability to attain or not attain certain clinical outcomes. The device can facilitate the acquisition of biometric data from the test subjects, as well as the aggregation of that data, in an effort to evaluate whether an experimental drug has therapeutic potential.

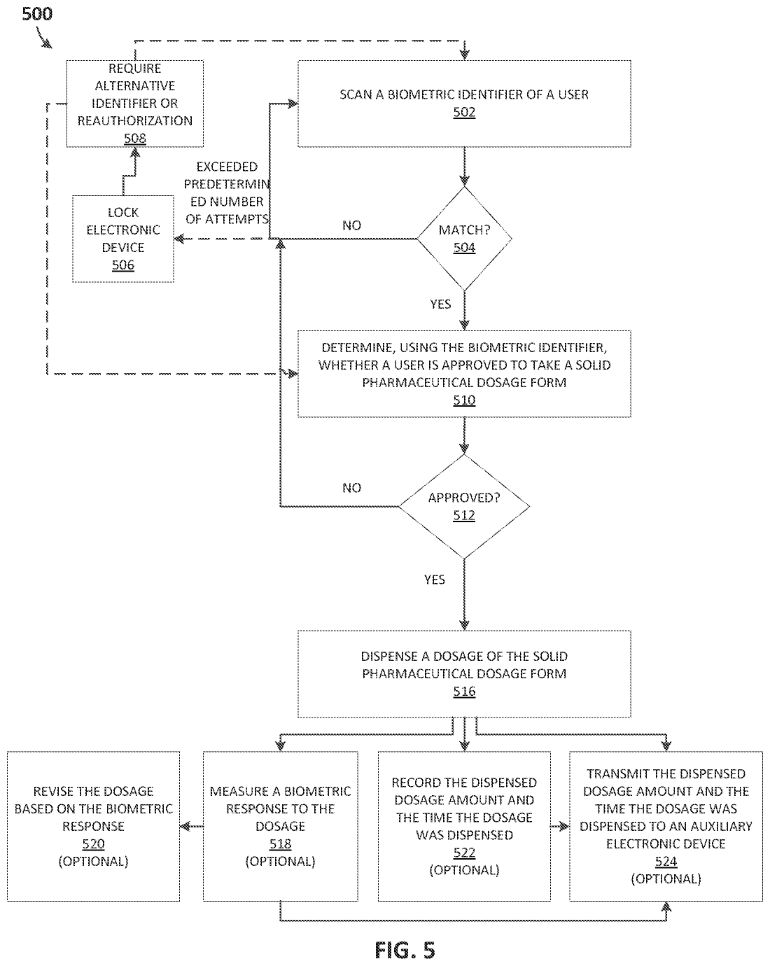

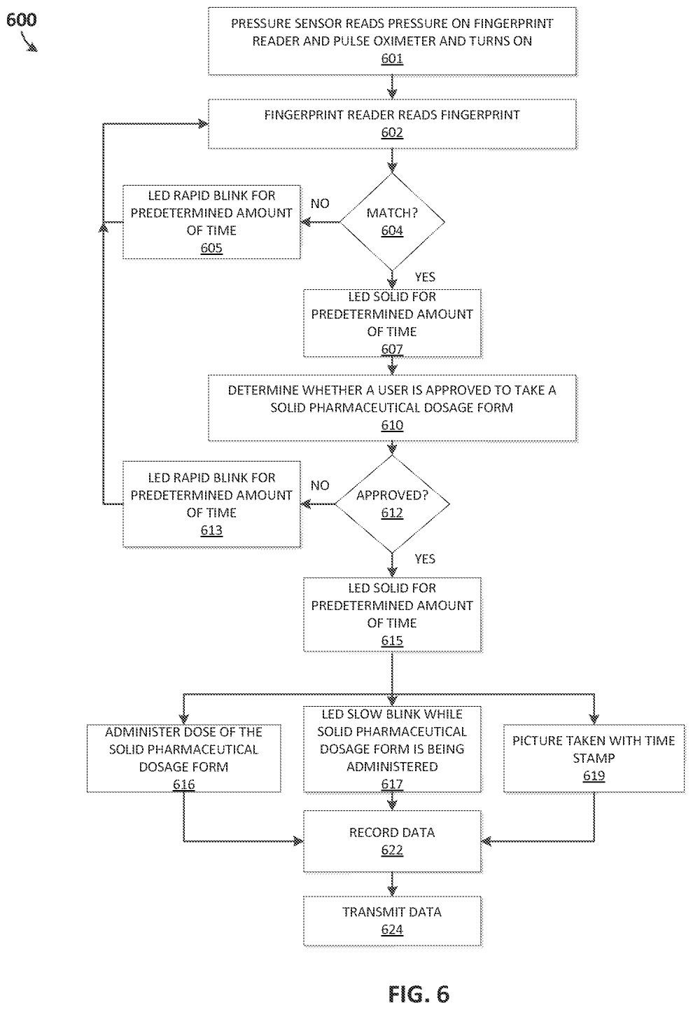

FIG. 5 is a representative flow diagram of an exemplary method 500 for administering a pharmaceutical or biological agent. In exemplary embodiments, the pharmaceutical agent delivery and biometric data acquisition device discussed throughout method 500 can have some or all of the same characteristics as the pharmaceutical agent delivery and biometric data acquisition device 100 described above in FIGS. 1-4. The pharmaceutical agent delivery and biometric data acquisition device will also be referred to herein as the “biometric data acquisition device.” For example, the pharmaceutical agent delivery and biometric data acquisition device can be turned on by holding a button (e.g., a fingerprint reader and/or pulse oximeter incorporated into the pharmaceutical agent delivery and biometric data acquisition device) for a predetermined amount of time, or by blowing air into or sucking air through the device. As another example, the pharmaceutical agent delivery and biometric data acquisition device can provide sensory feedback to a user intermittently during method 500. Examples of sensory feedback include, but are not limited to: visual cues, haptic feedback, or auditory feedback. As another example, the pharmaceutical agent delivery and biometric data acquisition device can take an image of the user intermittently during method 500. An example of sensory feedback is discussed in more detail in relation to FIG. 6 below. As another example, the pharmaceutical agent delivery and biometric data acquisition device can have wired and wireless connectivity. As another example, the pharmaceutical agent delivery and biometric data acquisition device can measure biometric responses of a user. This list, however, is not inclusive and, therefore, not meant to be limiting.

Method 500 begins by sensing a biometric identifier of a user using a pharmaceutical agent delivery and biometric data acquisition device (block 502). In exemplary embodiments, the biometric identifier includes, but is not limited to, the following: a fingerprint pattern, an iris pattern, a retina pattern, a vocal pattern, a facial-feature pattern, a pore pattern, a thermal image pattern, and a blood vessel pattern. The biometric data acquisition device can be equipped with various sensors and software to measure one or more of these biometric identifiers, as described above. In some embodiments, method 500 can begin when one or more audio sensors detects one or more audio signals from an authorized user, including the patient himself, or an authorized caregiver.

Next, at block 504, a determination can be made whether the scanned biometric identifier matches a stored biometric identifier. The stored biometric identifier can be an approved user's biometric identifier. In some exemplary embodiments, a stored biometric identifier can be securely stored in the pharmaceutical agent delivery and biometric data acquisition device's memory. Furthermore, in some exemplary embodiments, a stored biometric identifier can be concurrently securely stored on an auxiliary electronic device (e.g., a smartphone or a cloud computing device) that the pharmaceutical agent delivery and biometric data acquisition device can connect to, either wired or wirelessly. Or, in some other exemplary embodiments, the stored biometric identifier is not stored in the pharmaceutical agent delivery and biometric data acquisition device's memory, but only stored on an auxiliary electronic device, which the device can connect to, either wired or wirelessly. In exemplary embodiments, the stored biometric identifier can be included in a secure database of stored biometric identifiers. In exemplary embodiments, biometric identifiers for more than one user can be stored in the secure database of biometric identifiers and more than one biometric identifier for each user can be stored in the secure database of biometric identifiers.

In order to populate a list of stored biometric identifiers, an enrollment process can be undertaken. The enrollment process may include determining what biometric identifiers are to be used in method 500, enrolling each of those biometric identifiers using an iterative process, so that a fingerprint pattern, retina pattern, etc. can be recognized from various angles and under different conditions, and storing the enrollment data in the memory of the pharmaceutical agent delivery and biometric data acquisition device, or an auxiliary electronic device.

If the scanned biometric identifier matches a stored biometric identifier at block 504, then the method 500 continues to block 510. If the scanned biometric identifier does not match a stored biometric identifier, then method 500 can proceed back to scanning the biometric identifier at block 502. However, in some exemplary embodiments, if method 500 proceeds to scan a biometric identifier a predetermined number of times but is unable to match the scanned biometric identifier to a stored biometric identifier, then method 500 can proceed to locking the biometric data acquisition device at block 506. The predetermined number of times can be configurable when setting up the biometric data acquisition device. In some exemplary embodiments, method 500 will try to match the scanned biometric identifier to a stored biometric identifier three times before locking the biometric data acquisition device. In some other exemplary embodiments, method 500 will attempt to match the scanned biometric identifier to a stored biometric identifier five times before locking the biometric data acquisition device. In yet other exemplary embodiments, method 500 will attempt to match the scanned biometric identifier to a stored biometric identifier an unlimited number of times without locking the biometric data acquisition device.

In the embodiments where method 500 proceeds to block 506 and locks the biometric data acquisition device, method 500 can proceed to block 508 and require either an alternate identifier or reauthorization to unlock the pharmaceutical agent delivery and biometric data acquisition device. In some exemplary embodiments where method 500 requires an alternative identifier at block 508, a different biometric identifier than the one previously scanned may be scanned and matched to a stored biometric identifier in order to unlock the biometric data acquisition device. For example, if the biometric identifier initially scanned by the biometric data acquisition device was a fingerprint, then a user's retina may be scanned and matched to a stored biometric identifier in order to unlock the biometric data acquisition device. Or, as another exemplary embodiment, a passcode may be entered into the biometric data acquisition device in order to unlock the biometric data acquisition device. In other embodiments, block 508 may require reauthorization of the biometric data acquisition device by the manufacturer of the device, a certified healthcare professional or other third-party.

Once the biometric data acquisition device is unlocked, in some embodiments, method 500 can proceed back to block 502 or to block 510, depending on how the biometric data acquisition device was unlocked. For example, if a passcode was entered, method 500 may proceed back to 502 to identify a biometric identifier of a user since a biometric identifier was never matched to a stored biometric identifier. As another example, if a retina was scanned and the retina pattern is matched to a stored biometric identifier to unlock the biometric data acquisition device, method 500 may proceed to block 510 since a biometric identifier was matched to a stored biometric identifier. In yet another example, if the biometric data acquisition device was unlocked by the manufacturer, a healthcare professional or other third-party, the person or entity that unlocked the biometric data acquisition device may determine whether method 500 proceeds to block 502 or to block 510.

In embodiments where the scanned biometric identifier matches a stored biometric identifier at block 504, then method 500 determines, using the biometric identifier, whether the user is approved to take a pharmaceutical agent of a list of approved pharmaceutical agents for example, at block 510. In certain embodiments, determining whether the user is approved to take a pharmaceutical agent can include matching the scanned biometric identifier to a stored biometric identifier, wherein the stored biometric identifier is an approved user's biometric identifier. Additionally, in exemplary embodiments, each stored biometric identifier can be correlated to a user identifier of the user. The user identifier may be the name of the user, the social security number of the user or rendition thereof, a username of the user, or a random number assigned to the user during configuration of the pharmaceutical agent delivery and biometric data acquisition device. A random number or username may be used to protect the privacy of the user, in addition to the biometric identifier being correlated to a user identifier.

In addition to being correlated to a biometric identifier, the user can be linked to a list of pharmaceutical agents that are eligible to be taken by the user based on for example, medical history of the user. In exemplary embodiments, the list of pharmaceutical agents may include all the pharmaceutical agents currently and previously prescribed to the user of the user identifier. If the user has never been prescribed a pharmaceutical agent, the list of prescribed pharmaceutical agents can be the null set. In some embodiments, the list of prescribed pharmaceutical agents can be uploaded to the device by a healthcare professional. This can be done either remotely when the biometric data acquisition device is either wired or wirelessly connected to a network or when the biometric data acquisition device is in the presence of a healthcare professional. In some exemplary embodiments, the list of pharmaceutical agents may include over-the-counter pharmaceuticals, nutraceuticals, minerals, supplements, vitamins, and the like.

In addition to correlating a potential list of pharmaceutical agents that are available for use by the user (prescribed, monitored and non-prescribed) for a particular purpose or in general, determining whether the user affiliated with the user identifier is approved to take a target pharmaceutical agent may include determining the present time (realtime) and date during which the biometric identifier is scanned, when the last time or any previous time a biometric identifier was scanned or when the pharmaceutical agent was administered to the user and based in part on this information, whether the instant time and/or date is within a time period that the pharmaceutical agent is allowed, safe or optimal to be taken.

If a user is approved to take a pharmaceutical agent, then the biometric data acquisition device can give sensory feedback (e.g. visual or audio signal) to the user of the user identifier that the user is approved to take a pharmaceutical agent. In some exemplary embodiments, to determine whether a specific or family of pharmaceutical agents or monitored agents are approved to be taken by the user of the user identifier, the agent may be associated with the biometric data acquisition device by an authenticated user or an approved caregiver or healthcare provider. Then, the pharmaceutical agent delivery and biometric data acquisition device can determine whether the coupled pharmaceutical agent is approved to be taken by the user. The pharmaceutical agent delivery and biometric data acquisition device can determine what pharmaceutical agent is associated with the biometric data acquisition device. In accordance with these embodiments, this can be determined in a variety of ways including, but not limited to, radio-frequency identification (RFID), resistance sensing, barcode scanning, etc. In some other exemplary embodiments, to determine whether a specific pharmaceutical agent is approved to be taken by the user, the pharmaceutical agent delivery and biometric data acquisition device can include an input and sensory feedback device for selecting a specific or family of pharmaceutical agents from a list of pharmaceutical agents. Similar to blocks 502-510, sensory feedback can be given to the user throughout the process of determining whether a user is approved to take a pharmaceutical agent.

In some embodiments, stored biometric information can be used to determine whether one or more of a subject's current biometrics is anomalous or abnormal and whether this observation can be connected to administration of a particular pharmaceutical, monitored or biological agent. For example, a subject's biometric data can be acquired and stored on the device or an auxiliary electronic device. If a subject's specific instantaneous biometric response is outside a certain usage range, which has been established by the subject's recent history of biometric responses aggregated together, an alarm may be triggered by the device, even if the biometric response has been determined to be within an acceptable, previously determined range (e.g., a clinical range determined from patient trials). In this way, the device can be customized to operate according to a subject's individualized biometric responses.

At block 512, for example, if the user identifier is approved to take a pharmaceutical agent of interest to treat a disease or condition, method 500 proceeds to block 514 or block 516. On the other hand, in embodiments, if the user identifier is not approved to take the pharmaceutical agent of interest, then method 500 proceeds to block 502 or the method 500 ends. Similar to the blocks above, sensory feedback can be given to the user as to whether method 500 is proceeding to block 502, block 514, block 516, or method 500 is ending. Depending on the feedback, a user where the pharmaceutical agent is not approved can receive feedback to contact their physician or seek alternative assistance.

In other exemplary embodiments, if a solid pharmaceutical dosage form is approved at block 512 method 500 proceeds from block 512 to block 516. At block 516, a dosage of the solid pharmaceutical dosage form can be dispensed. In some exemplary embodiments, dispensing a dosage of the solid pharmaceutical dosage form includes, but is not limited to, identifying a solid pharmaceutical dosage form associated with the biometric data acquisition device; and determining whether the solid pharmaceutical dosage form is the same as the approved solid pharmaceutical dosage form.

In another exemplary embodiment, dispensing a dosage can include identifying a solid pharmaceutical dosage form associated with the biometric data acquisition device; determining whether the solid pharmaceutical dosage form is the same as the approved solid pharmaceutical dosage form; and dispensing a dosage of the solid pharmaceutical dosage form through the loading cartridge of the device. In some embodiments, a method can further include repeating this process for all subsequent dosages dispensed to a subject, including revised and unrevised dosages, as well as recording the biometric data associated with the subsequent dosages to evaluate, for example, whether the patient is or is not complying a with a certain treatment plan.

In some embodiments, method 500 may continue to block 518 and measure a biometric response to the dosage dispensed. In some embodiments, the biometric response of the user can measured relatively soon after the solid pharmaceutical dosage form has been dispensed or after a period have time has lapsed. Or, in some embodiments, the biometric response of the user can be done periodically. In other embodiments, the biometric data acquisition device can be equipped with various sensors to measure one or more of the following biometric responses: a galvanic skin response, a blood oxygen level response, a body temperature response, a heartrate response, a perfusion index, a blood pressure response, a retina response, an eye movement response, eye color (e.g., yellowing of the sclera), an inhalation velocity response, an inhalation pressure response, an inhalation volume response, an expiratory velocity response, an expiratory pressure response, an expiratory volume response, or an exhale chemical composition response. This list, however, is not inclusive and, therefore, is not meant to be limiting.

In some embodiments where a biometric response can be measured by the biometric data acquisition device, method 500 can proceed to block 520 where the dosage is updated to generate a revised dosage based on the measured biometric response. In exemplary embodiments, the dosage can be revised by a healthcare professional after the information regarding a previous dosage, the time of a previous dosage, the frequency of a previous dosage and the biological response to a previous dosage has been transmitted to the healthcare professional.

In other embodiments, method 500 may continue to block 522 and record, on the biometric data acquisition device's memory, each time a dosage was dispensed, wherein the amount of the dosage, the time of day the dosage was dispensed and the date of the dosage was dispensed. In certain examples, all dosages subsequent to a dosage, including revised and unrevised dosages, can be recorded and used to evaluate a subject's cooperation with a pre-determined treatment plan. In some embodiments, this information may be transmitted to an auxiliary electronic device.

For each of blocks 516, 518, 522, amount of administered dosage, time of the dosage, frequency of the administered dosage, whether or not the dosage was revised, and any other information that may be pertinent in order to monitor treatment of the user can be transmitted to an auxiliary electronic device. The information can be transferred using a wired connection or a wireless connection if and when one becomes available. In some embodiments, until a network connection becomes available, the information can be stored on the pharmaceutical agent delivery and biometric data acquisition device's memory.

FIG. 6 is a flow diagram representing a method 600 illustrating one example of method 500. Method 600 serves as an example and is not meant to be limiting. In embodiments where a user is visually impaired, any visual cues (e.g., the LED lights) in method 600 can be replaced with other sensory feedback (e.g., auditory or haptic feedback). Method 600 begins by turning on the pharmaceutical agent delivery and biometric data acquisition device at block 601. In some embodiments, this can be done by holding down on a fingerprint reader and pulse oximeter included in the pharmaceutical agent delivery and biometric data acquisition device for a predetermined amount of time. For example, holding down on the fingerprint reader and pulse oximeter for 5 seconds may turn the pharmaceutical agent delivery and biometric data acquisition device on.

Once the pharmaceutical agent delivery and biometric data acquisition device is on, method 600 can proceed to block 602 where a fingerprint of a user is scanned by a fingerprint scanner that can be included in the pharmaceutical agent delivery and biometric data acquisition device. At block 604, if the scanned fingerprint does not match a stored fingerprint, then method 600 proceeds to block 605, at which time an indicator, such as a rapidly blinking LED notifies the user that the scanned fingerprint did not match a stored fingerprint. Method 600 then proceeds back to block 602 to allow the user to scan their fingerprint again. If, however, the scanned fingerprint matches a stored fingerprint, then method 600 proceeds to 607, at which time a different indicator, such as a solidly illuminated LED notifies the user that their scanned fingerprint matches a stored fingerprint.

At block 610, method 600 proceeds by determining (e.g. by stored information) whether the user correlated to the scanned and matched fingerprint is approved to take a particular pharmaceutical or other monitored agent. As detailed above, this may include determining the current time and date, when the last time the pharmaceutical agent was administered to the user and whether the current time and date is within an allowable or recommended time period for the user to administer a dose of the pharmaceutical agent.

At block 612, if a determination is made that the user is not approved to take a pharmaceutical agent, then method 600 can proceed to block 613, at which time an indicator such as a rapidly blinking LED or audio notifies the user that the a pharmaceutical agent has not been approved for the user to take at the time of the fingerprint read. If method 600 does proceed to block 613 because the user is not approved to take a pharmaceutical agent, then method 600 can revert back to block 602 or method 600 can end. If, a determination is made that the user being assessed is approved to take a pharmaceutical or other monitored agent at block 612, then method can proceed to block 615, at which time an indicator such as a solidly illuminated LED or audio signal notifies the user that a pharmaceutical agent has been approved to be taken by the user.

After block 615, method 600 proceeds to blocks 616, 617, and/or 619 which can occur concurrently or within a prescribed time period of one another. At block 616, a dosage of the solid pharmaceutical agent can be administered by the pharmaceutical agent delivery and biometric data acquisition device. The dosage can be administered as describe above in method 500. While the pharmaceutical agent is being administered, and in some embodiments before, an LED or audio can slowly blink or sound-off to notify the user that the pharmaceutical agent is being administered or about to be administered at block 617. When the administration of the pharmaceutical agent is completed, the LED can stop blinking or the audio shuts off. Concurrently with the administration of the pharmaceutical agent, the pharmaceutical agent delivery and biometric data acquisition device can be configured to take a picture (e.g. for identification or assessment of after effects etc.) of the user at block 619. In some embodiments, a timestamp can be included with the picture, so that the time that the pharmaceutical agent was administered can be recorded along with a record number and one or more user identifiers (e.g., user's picture).

In another embodiment, method 600 can then proceed to block 622 where the data from administration can be recorded to memory pharmaceutical agent delivery and biometric data acquisition device. In some embodiments, the data recorded can be any of the data discussed above in method 500. Examples include, but are not limited to, pharmaceutical or monitored agent, dose administered of the pharmaceutical agent, time and date of the administration of the pharmaceutical agent, and the biometric response to the administration of the biological agent. In other embodiments, other agents (e.g. over-the-counter agents, vitamins) taken or used by a user can also be recorded by the user using a recorder on the device or other method for recordation in realtime or at a later time by the user.

Method 600 can proceed to block 624 where the recorded data can be transmitted to a secondary electronic device, a cloud computing device or an application stored therein while backed-up by the device. Various user identifiers can be associated with a user's biometric data stored on an electronic record, such that the user can access the biometric data using his/her user identifier. In some embodiments, the user's biometric data can be uploaded and stored in a cloud computing device that can be accessed using one or more user identifiers

A number of variations and modifications of the disclosure can be used. It would be possible to provide for some features of the disclosure without providing others.

For example, systems and methods of this disclosure can be implemented in conjunction with a special purpose computer, a programmed microprocessor or microcontroller and peripheral integrated circuit element(s), an ASIC or other integrated circuit, a digital signal processor, a hard-wired electronic or logic circuit such as discrete element circuit, a programmable logic device or gate array such as PLD, PLA, FPGA, PAL, special purpose computer, any comparable means, or the like. In general, any device(s) or means capable of implementing the methodology illustrated herein can be used to implement the various aspects of this disclosure. Exemplary hardware that can be used for the disclosed embodiments, configurations and aspects includes computers, handheld devices, telephones (e.g., cellular, Internet enabled, digital, analog, hybrids, and others), and other hardware known in the art. Some of these devices include processors (e.g., a single or multiple microprocessors), memory, nonvolatile storage, input devices, and output devices. Furthermore, alternative software implementations including, but not limited to, distributed processing or component/object distributed processing, parallel processing, or virtual machine processing can also be constructed to implement the methods described herein.

In yet another embodiment, the disclosed methods may be readily implemented in conjunction with software using object or object-oriented software development environments that provide portable source code that can be used on a variety of computer or workstation platforms. Alternatively, the disclosed system may be implemented partially or fully in hardware using standard logic circuits or VLSI design. Whether software or hardware can be used to implement the systems in accordance with this disclosure can be dependent on the speed and/or efficiency requirements of the system, the particular function, and the particular software or hardware systems or microprocessor or microcomputer systems being utilized.

In yet another embodiment, the disclosed methods may be partially implemented in software that can be stored on a storage medium, executed on programmed general-purpose computer with the cooperation of a controller and memory, a special purpose computer, a microprocessor, or the like. In these instances, the systems and methods of this disclosure can be implemented as program embedded on personal computer such as an applet, JAVA® or CGI script, as a resource residing on a server or computer workstation, as a routine embedded in a dedicated measurement system, system component, or the like. The system can also be implemented by physically incorporating the system and/or method into a software and/or hardware system.

The present disclosure, in various aspects, embodiments, and configurations, includes components, methods, processes, systems and/or apparatus substantially as depicted and described herein, including various aspects, embodiments, configurations, sub combinations, and subsets thereof. Those of skill in the art will understand how to make and use the various aspects, aspects, embodiments, and configurations, after understanding the present disclosure. The present disclosure, in various aspects, embodiments, and configurations, includes providing devices and processes in the absence of items not depicted and/or described herein or in various aspects, embodiments, and configurations hereof, including in the absence of such items as may have been used in previous devices or processes, e.g., for improving performance, achieving ease and\or reducing cost of implementation.

The foregoing discussion of the disclosure has been presented for purposes of illustration and description. The foregoing is not intended to limit the disclosure to the form or forms disclosed herein. In the foregoing Detailed Description for example, various features of the disclosure are grouped together in one or more, aspects, embodiments, and configurations for the purpose of streamlining the disclosure. The features of the aspects, embodiments, and configurations of the disclosure may be combined in alternate aspects, embodiments, and configurations other than those discussed above. This method of disclosure is not to be interpreted as reflecting an intention that the claimed disclosure requires more features than are expressly recited in each claim. Rather, as the following claims reflect, inventive aspects lie in less than all features of a single foregoing disclosed aspects, embodiments, and configurations. Thus, the following claims are hereby incorporated into this Detailed Description, with each claim standing on its own as a separate preferred embodiment of the disclosure.

Moreover, though the description of the disclosure has included description of one or more aspects, embodiments, or configurations and certain variations and modifications, other variations, combinations, and modifications are within the scope of the disclosure, e.g., as may be within the skill and knowledge of those in the art, after understanding the present disclosure. It is intended to obtain rights which include alternative aspects, embodiments, and configurations to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. A solid pharmaceutical agent dosage form dispensing and biometric data acquisition device comprising:
a housing unit;
a power source;
a processor and memory;
a loading cartridge comprising a plurality of rotatable storage chambers for storing and dispensing one or more solid pharmaceutical dosage forms;
a prime mover operably coupled to the loading cartridge and a dispensing element, wherein activation of the dispensing element actuates the prime mover to eject one or more solid pharmaceutical agent dosage forms from the loading cartridge;
at least one scanner operatively coupled to the processor, wherein the processor verifies an identify of a subject based upon information obtained by the at least one scanner;
at least one biometric sensor for sensing biometric data of the subject, wherein the biometric data is sensed and acquired during at least one of prior to, during, and after dispensing the one or more solid pharmaceutical dosage forms to the subject, wherein the biometric data is stored in memory; and
at least one data transfer port.

2. The device according to claim 1, wherein the device further comprises an airflow port and an image acquisition device centrally aligned with the airflow port, the image acquisition device comprising a lens, an image sensor and signal wires which operatively connect the image acquisition device to the processor; and
at least one visual indicator that emits at least one light signal operatively coupled to the image acquisition device and facing the same direction as the lens.

3. The device according to claim 2, further comprising an airflow port accessory module, wherein the airflow port accessory module is coupled to the airflow port to facilitate the inhalation and exhalation of the user's breath into the airflow port.

4. The device according to claim 1, wherein the at least one biometric sensor includes one or more of a temperature sensor, a thermal imaging sensor, a galvanic skin response sensor, a pulse oximeter, a carbon dioxide sensor, an oxygen sensor, an optical sensor, an air flow velocity sensor, an air pressure sensor, a chemical sensor, and a global positioning system (GPS) sensor.

5. The device according to claim 1, wherein the device further comprises one or more peripheral module interfaces for coupling one or more peripheral modules to the device.

6. The device according to claim 5, wherein the one or more peripheral modules includes one or more of a blood pressure monitor, a blood glucose monitor, a CPAP machine, an electrocardiogram device, a battery, and a battery charger.

7. The device according to claim 1, wherein the at least one scanner comprises at least one of a fingerprint reader or a pressure sensor.

8. (canceled)

9. The device according to claim 1, wherein the device further comprises at least one of a radio-frequency identification (RFID) reader or Bluetooth hardware and software components.

10. (canceled)

11. The device according to claim 1, wherein the processor further comprises one or more software programs for operating the at least one biometric sensor and for facilitating the acquisition of biometric data using the at least one biometric sensor.

12. The device according to claim 1,wherein the one or more solid pharmaceutical dosage form is one or more of a pill, capsule, gel capsule, tablet, caplet, patch, strip, and thin-film strip.

13. A system for dispensing a solid pharmaceutical agent dosage form to and acquiring biometric data from a subject, the system comprising:
a housing unit;
a power source;
a processor and memory;
a loading cartridge comprising a plurality of rotatable storage chambers for storing and dispensing one or more solid pharmaceutical agent dosage forms;
a prime mover operably coupled to the loading cartridge and a dispensing element, wherein activation of the dispensing element actuates the prime mover to eject one or more solid pharmaceutical dosage forms from the loading cartridge;
at least one scanner operatively coupled to the processor, wherein the processor verifies an identify of a subject based upon information obtained by the at least one scanner;
at least one biometric sensor for sensing biometric data of the subject, wherein the biometric data is sensed and acquired during at least one of prior to, during, and after dispensing the one or more solid pharmaceutical dosage forms to the subject, wherein the biometric data is stored in memory; and
at least one data transfer port;
wherein the system facilitates the dispensing of the one or more solid pharmaceutical dosage forms to the subject, and the acquisition of the biometric data from the subject.

14-23. (canceled)

24. The system according to claim 13, wherein the one or more solid pharmaceutical dosage form is one or more of a pill, capsule, gel capsule, gummy, timed-release capsule, slow-dissolving capsule, tablet, caplet, patch, strip, and thin-film strip , mini-tablets, granules, beads, pellets, multiparticulates, spheroids, or combinations thereof

25. A method for dispensing a solid pharmaceutical agent dosage form to an authorized user, the method comprising:
scanning a biometric identifier of a user using a biometric data acquisition and solid pharmaceutical dosage form dispensing device;
determining, using the biometric identifier, whether the user is approved to take a dosage of a solid pharmaceutical dosage form; and
dispensing the dosage of the solid pharmaceutical agent dosage form from the solid pharmaceutical agent dosage form dispensing and biometric data acquisition device, if the user is approved to take the dosage of the solid pharmaceutical dosage form.

26. The method according to claim 25, wherein the biometric identifier includes at least one of the following: a fingerprint pattern, an iris pattern, a retina pattern, a vocal pattern, a facial-feature pattern, a pore pattern, a thermal image pattern, or a blood vessel pattern.

27. The method according to claim 25, wherein determining whether the user is approved to take a dosage of a solid pharmaceutical agent dosage form comprises:
matching the scanned biometric identifier to a stored biometric identifier, wherein the stored biometric identifier is an approved user's biometric identifier;
identifying if the solid pharmaceutical agent dosage form is included on a list of solid pharmaceutical dosage forms that are eligible to be taken by the user;
identifying a recent time that the biometric identifier was scanned and the solid pharmaceutical agent dosage form was dispensed; and
approving the dispensing of the solid pharmaceutical dosage form if a present time during which the biometric identifier is scanned is within an approved time period for dispensing the solid pharmaceutical dosage form, based on the recent time that the biometric identifier was scanned.

28. The method according to claim 25, further comprising recording, on the solid pharmaceutical agent dosage form and biometric data acquisition device's memory, times that the solid pharmaceutical dosage form is dispensed.

29. (canceled)

30. The method according to claim 25, further comprising taking an image of the user, using an imaging device, when dispensing a dosage of the solid pharmaceutical agent dosage form, wherein the imaging device is coupled to the biometric data acquisition and solid pharmaceutical agent dosage form dispensing device.

31-32. (canceled)

33. The method according to claim 25, further comprising measuring a biometric response to the dosage of the solid pharmaceutical dosage form.

34. (canceled)

35. The method according to claim 33, further comprising transmitting the dosage of the solid pharmaceutical dosage form and the measured biometric response to an auxiliary electronic device.

36. The method according to claim 33, further comprising dispensing a revised dosage of the solid pharmaceutical agent dosage form based on the measured biometric response.

37-38. (canceled)
